(19) United States
(12) Patent Application Publication
(10) Pub. No.: US 2010/0305563 A1
(43) Pub. Date: Dec. 2, 2010

(54) ELECTROSURGICAL SYSTEM

(76) Inventor: Kelvin J. VARNEY, Monmouthshire (GB)

Correspondence Address: NIXON & VANDERHYE, PC, 901 NORTH GLEBE ROAD, 11TH FLOOR, ARLINGTON, VA, 22203, US

(21) Appl. No.: 12/784,994

(22) Filed: May 21, 2010

Related U.S. Application Data
(60) Provisional application No. 61/213,381, filed on Jun. 3, 2009.

(30) Foreign Application Priority Data
May 29, 2009 (GB) 0909322.0

(51) Int. Cl. A61B 18/14

(52) U.S. Cl. 606/41

(57) ABSTRACT

An electrosurgical system includes a first component (11) and a second component (7) detachably connected one to the other. The first component is provided with a socket (33) including a plurality of contacts (34 to 40), and the second component is provided with a plug (17) including one or more pins (19). The arrangement is such that when the plug (17) is received within the socket (33) the one or more pins (19) make electrical contact with a particular combination of the plurality of contacts (34 to 40). The electrosurgical system is provided with means for identifying the second component (7) by determining the combination of contacts (34 to 40) in which the one or more pins (19) are in electrical contact.

This invention relates to an electrosurgical system for use in the treatment of tissue. Such systems are used in endoscopic or “keyhole” surgery, as well as more traditional “open” surgery.

Many electrosurgical systems have some form of identification system, such that when an electrosurgical instrument is connected to an electrosurgical generator, the generator is able to detect which type of instrument is present, and even use settings such as power and voltage settings which are appropriate for that particular instrument or type of instrument. Our U.S. Pat. No. 6,074,386 is one example of such an identification system, although other types are also known. The present invention attempts to provide an improved alternative identification system, capable of identifying different electrosurgical instruments to the generator.

Accordingly, an electrosurgical system is provided including a first component and a second component detachably connected one to the other, the first component being provided with a socket including a plurality of contacts, and the second component being provided with a plug including one or more pins, the arrangement being such that when the plug is received within the socket the one or more pins make electrical contact with a particular combination of the plurality of contacts, the electrosurgical system being provided with means for identifying the second component by determining the combination of contacts in which the one or more pins are in electrical contact, wherein the plug includes at least one pin provided with different longitudinal portions, wherein each longitudinal portion along the pin is either provided with a first condition such that it makes electrical contact with its respective contact within the socket, or a second condition such that it does not make electrical contact with its respective contact within the socket, and wherein the first and second conditions are constituted by a different dimension for the longitudinal portions.

The one or more pins of the plug are designed to make electrical contact with a predetermined combination of the contacts on the socket, which combination can be determined to identify the instrument being utilized. By providing plugs which make contact with different combinations of contacts, different types of instrument can be easily recognized by the generator.

The first and second conditions are constituted by a different dimension for the longitudinal portions. For example, the length of one or more conductive portions can be varied between different instruments, and detected in order to identify those instruments. Alternatively, the first and second conditions are conceivably constituted by a radius for the longitudinal portions. In this way, relatively wide portions do make contact with the corresponding contacts within the socket, whereas other relatively narrow portions do not. By varying the combination of the wide and narrow portions, different instruments can be easily distinguished from one another. The different dimension need not necessarily be the radius of the pin, for the pin need not be of circular cross-section. As an alternative example of a different dimension, the pin may be provided with recessed or cut-out portions, so as to provide some areas in contact with respective socket contacts, and other areas not in contact.

Conveniently, some or all of the plurality of contacts within the socket are connected to an identification component. This identification component is conceivably a circuit adapted to generate an identifiable characteristic signal. An example of such a circuit is a memory device such as a digital memory device. Additionally or alternatively the identification component is conveniently a passive electrical identification component having a parameter of a finite non-zero value. Where the identification component is a passive identification component with a non-zero value, the identifying means conveniently identifies the second component by determining the total value of the passive electrical identification components for those contacts within the socket to which the one or more pins are in electrical contact. In one arrangement the passive identification components are each of the same value, and the total value determines how many identification components are in electrical connection. However, in a preferred alternative arrangement, the passive identification components each have different values. In this way, the permitted total values for the identification components can be set as predetermined discrete total values, thereby distinguishing between a correct authorized total and a false total caused by an incorrect or shorted connection between one the contacts.

In one typical arrangement the passive electrical identification component is a resistor and the parameter is resistance. The identifying means totals the resistances attached to all those contacts within the socket that are in electrical contact with the one or more pins to establish a total resistance which is then used to identify the instrument concerned. Different types of instrument have different pins, with different combinations in contact with the contacts within the socket. Thus, for these different types of instrument, the total resistance established by the identifying means will be a different value, thereby distinguishing the instrument as belonging to a different category. As the vast majority of electrosurgical systems use alternating current power supplies, for the avoidance of doubt, the term “resistance” includes the resistive component of any ac circuit, forming part of the overall impedance.

In an alternative arrangement, the passive electrical identification component is conceivably a capacitor and the parameter is capacitance. As with the example given above, the identifying means totals the capacitors attached to all those contacts within the socket that are in electrical contact with the one or more pins to establish a total capacitance which is then used to identify the instrument concerned. As in our previous U.S. Pat. No. 6,074,386, one way in which the total capacitance can be established is by setting up a resonant circuit, the frequency of which provides an indication of the total capacitance and hence the identity of the instrument concerned. As before, for the avoidance of doubt, the term “capacitance” includes the capacitive component of any ac circuit, forming part of the overall impedance.

The present invention can be used in electrosurgical systems employing different arrangements. Electrosurgical systems differ in that some have a unitary handpiece, while others have a separate handpiece and electrosurgical assembly. Some handpieces have unitary cables, while others have detachable cables. Conveniently, the first component is selected from the group comprising electrosurgical generator, cable, or electrosurgical handpiece, while the second component is selected from the group comprising cable, electrosurgical handpiece, or electrode assembly. Thus, it can be seen that the plug and socket arrangement described herein can be used to connect a cable to an electrosurgical generator, a cable to an electrosurgical handpiece, or an electrode assembly to an electrosurgical handpiece. Whichever arrangement is used, the combination of contacts identifies the second component to the first component, thereby establishing which type of electrosurgical instrument, is being utilized in combination with the electrosurgical generator.

The present invention offers the advantage that if an identifying component (such as a resistor, capacitor or digital memory) is present, it does not necessarily need to be physically associated with the element being identified. For example, where a treatment electrode is the element to be identified, there is often limited physical space to allow for a resistor, capacitor or digital memory chip to be associated with the electrode. One solution is to locate these components in another part of the system, such as a cable or connector, but this means that in effect it is the cable or connector, rather than the electrode, which is being identified by the system. This means that there is the possibility that the “wrong” electrode can be employed with the cable or connector, and be incorrectly identified by means of the identifying components located in the cable or connector. In contrast, the present invention allows for identifying components to be located elsewhere in the system, and yet it is the combination provided by the plug or pin(s) directly associated with the treatment electrode that provides the identification. Thus, the electrode is identified by means of the contacts associated with the electrode, even if the identification components (resistors, capacitors or digital memory chip) are located elsewhere in the system.

Where space is an issue, and yet sophisticated identification is still required, a two-part system can be used. In this, a first identification element (such as a resistor or capacitor) is provided on the electrode assembly, and a second identification element (such as a digital memory chip) is provided elsewhere on the system, such as in a cable or connector. The first identification element is sufficiently small so as to be carried on the electrode assembly, and provides basic identification (manufacturer, or type of electrode, etc.). The second identification element does not need to be directly carried on the electrode assembly, and can provide more sophisticated identification, such as specific electrode identification, or parameters for the energy to be supplied by the generator etc. The second identification element can even update the generator in respect of the parameters for the energy to be supplied to electrode assemblies other than the one currently identified, i.e. other instruments in the range.

According to a further aspect of the invention, an electrosurgical system is provided including a first component and a plurality of second components each capable of being individually connected to the first component, the first component being provided with a socket including a plurality of contacts, and the second component being provided with a plug including one or more pins, the arrangement being such that when the plug is received within the socket the one or more pins make electrical contact with a particular combination of the plurality of contacts, the electrosurgical system being provided with means for identifying the second component by determining the combination of contacts in which the one or more pins are in electrical contact, wherein the plug includes at least one pin provided with different longitudinal portions, wherein each longitudinal portion along the pin is either provided with a first condition such that it makes electrical contact with its respective contact within the socket, or a second condition such that it does not make electrical contact with its respective contact within the socket, and wherein the first and second conditions are constituted by a different dimension for the longitudinal portions.

According to a still further aspect of the present invention, an electrosurgical system is provided including a first component and a plurality of second components each capable of being individually connected to the first component, the second components being provided in a plurality of different types of second component, the first component being provided with a socket including a plurality of contacts, and the second component being provided with a plug including one or more pins, the arrangement being such that when the plug is received within the socket the one or more pins make electrical contact with a particular combination of the plurality of contacts, the electrosurgical system being provided with means for identifying the type of the second component by determining the combination of contacts in which the one or more pins are in electrical contact, wherein the plug includes at least one pin provided with different longitudinal portions, wherein each longitudinal portion along the pin is either provided with a first condition such that it makes electrical contact with its respective contact within the socket, or a second condition such that it does not make electrical contact with its respective contact within the socket, and wherein the first and second conditions are constituted by a different dimension for the longitudinal portions.

In an alternative arrangement, an electrosurgical system is provided including a first component and a second component detachably connected one to the other, the first component being provided with a socket including a plurality of contacts, and the second component being provided with a plug including a plurality of pins, the arrangement being such that when the plug is received within the socket the plurality of pins make electrical contact with a particular combination of the plurality of contacts, the electrosurgical system being provided with means for identifying the second component by determining the combination of contacts in which the plurality of pins are in electrical contact.

Each pin within the plug is conveniently either provided with a first condition such that it makes electrical contact with its respective contact within the socket, or a second condition such that it does not make electrical contact with its respective contact within the socket. For example, in one arrangement the first and second conditions are constituted by a different length for the pins. Long pins make contact with the contacts within the socket, whereas other shorter pins do not. By varying the combination of short and long pins, different instruments can be identified.

Alternatively, the first and second conditions are constituted by providing the pin with either an electrically conductive or electrically non-conductive contact portion. For example, some pins can have conductive end portions, while other pins have non-conductive end portions, or end portions that are masked with an electrically insulating material. By varying the pins that are non-conductive, once again different instruments can be identified.

Each pin can conceivably have two portions, one designed to make contact with a first set of contacts within the socket, and the other designed to make contact with a second set of contacts within the socket. For example, the first set of contacts can be used for supplying electrosurgical voltage signals, data or other signals, while the second set of contacts can be used for identifying the electrosurgical instrument. This way, all of the pins are utilized, even though not all make contact with the second set of contacts. Alternatively, a relatively large number of pins can be provided, such that all of the functions of the electrosurgical system can be performed even though selected pins are not utilized.

The invention will now be described in more detail, by way of example only, with reference to the accompanying drawings, in which;

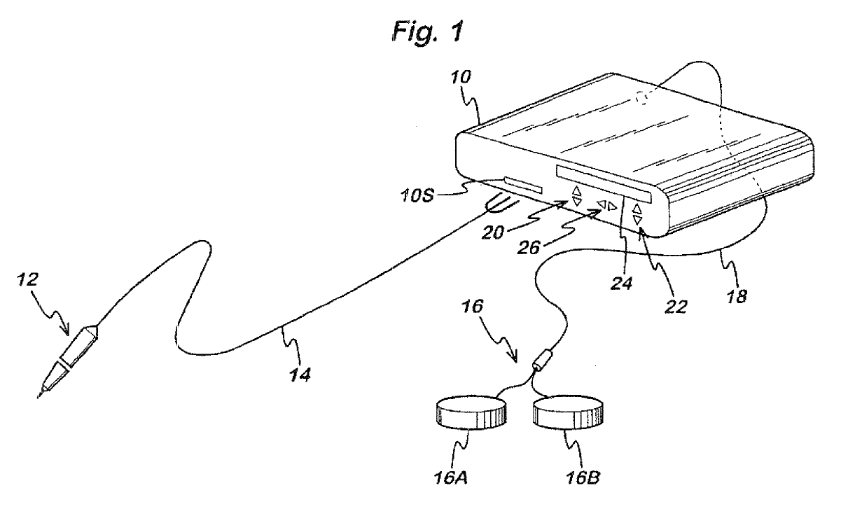

FIG. 1 is a schematic diagram of an electrosurgical system in accordance with the present invention,

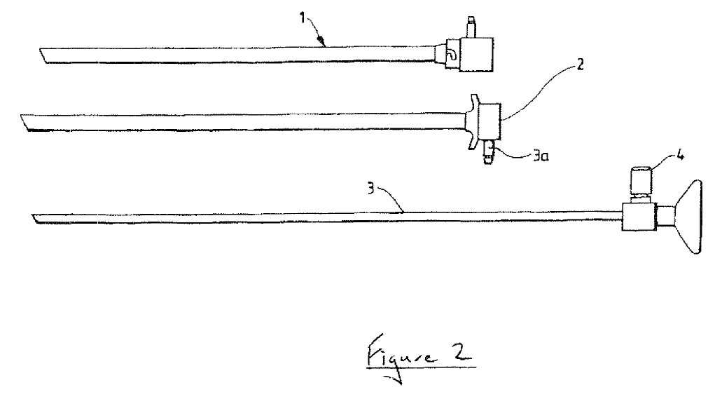

FIG. 2 is a side view of particular components making up the instrument which is a part of the system of FIG. 1,

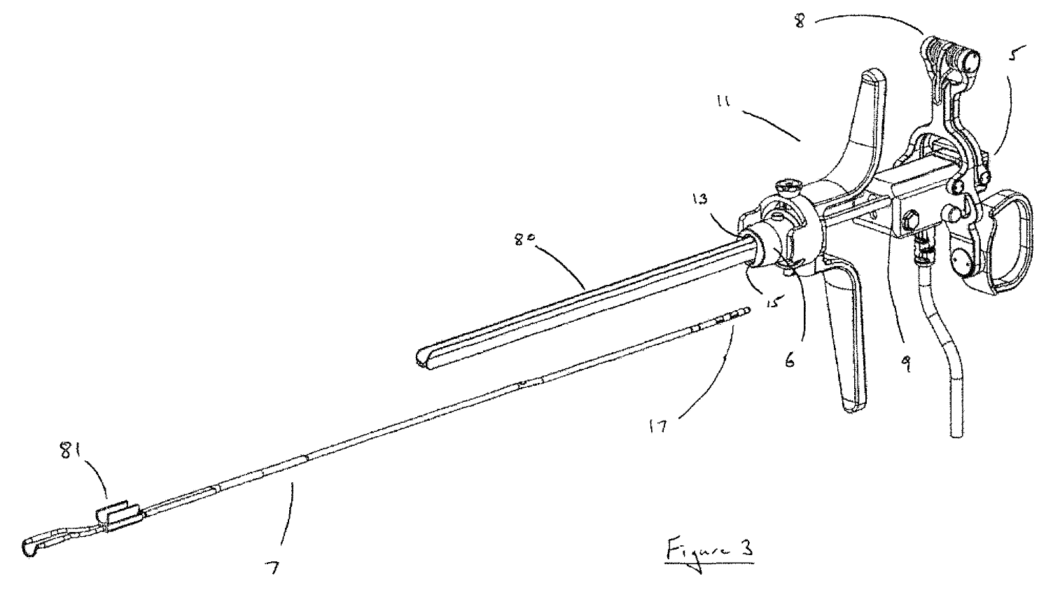

FIG. 3 is a perspective view of other particular components making up the instrument which is a part of the system of FIG. 1,

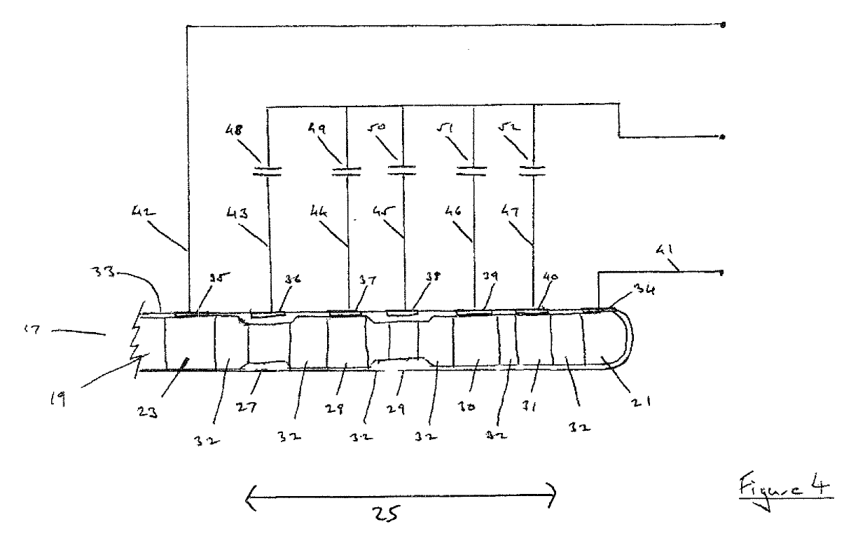

FIG. 4 is a schematic diagram of a plug and socket arrangement connecting two of the components of FIG. 3, and

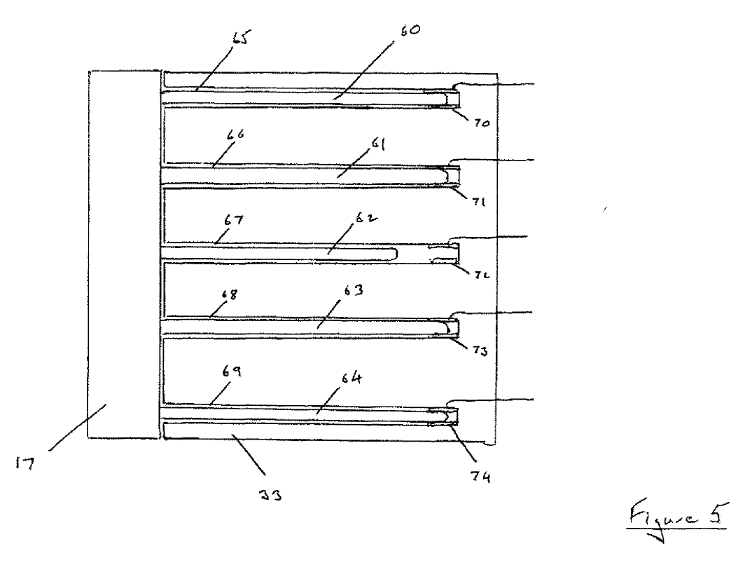

FIG. 5 is a schematic diagram of an alternative embodiment of plug and socket arrangement connecting two of the components of FIG. 3.

Referring to FIG. 1, a generator 10 has an output socket 10S providing a radio frequency (RF) output for an instrument 12 via a connection cord 14. Activation of the generator may be performed from the instrument 12 via a connection in cord 14 or by means of a footswitch unit 16, as shown, connected to the rear of the generator by a footswitch connection cord 18. In the illustrated embodiment footswitch unit 16 has two footswitches 16A and 16B for selecting a coagulation mode and a cutting mode of the generator respectively. The generator front panel has push buttons 20 and 22 for respectively setting coagulation and cutting power levels, which are indicated in a display 24. Push buttons 26 are provided as a means for selection between alternative coagulation and cutting waveforms.

FIGS. 2 & 3 show an embodiment of the instrument 12 in more detail. As shown in FIGS. 2 & 3, the instrument is in the form of a resectoscope consisting of several main components, an inner sheath 1, an outer sheath 2, a rod lens telescope/light source assembly 3, a working element 11, and a bipolar electrode assembly 7. The sheaths 1 and 2 provide for the supply and aspiration of an operating site with a fluid medium via a connector 3a. The outer sheath 2 locks over the inner sheath 1, forming a watertight seal. Typically, the inner sheath 1 has a diameter of 24βr, and the outer sheath 2 has a diameter of 27 Fr. The telescope assembly 3 provides the means of illuminating and viewing the operative site via a light source (not shown) connected thereto by a connector 4. The viewing angle of the telescope is generally at 30° to its axis.

The working element 11 may be either passive or active, that is to say the cutting stroke of the electrode may be as the result of a spring bias or against the force of a spring bias. The telescope assembly 3 is received within the working assembly 11 by means of a telescope connector 5 at its proximal end. The telescope assembly passes through a sealing block 6 having an aperture 13 therein, the inner sheath 1 also being connected to the sealing block. Both of these interfaces are watertight. A support guide 80 is provided on the distal side of the sealing block 6. Two spring-loaded links 8 and an insulation block 9, located between the sealing block 6 and the telescope connector 5, make up the mechanism. The active mechanism is arranged so that the spring-loaded links 8 assist the forward stroke, while, in the passive version the links aid the backward stroke. In general, the range of travel is about 25 mm.

An aperture 13 in the sealing block 6 allows the telescope support assembly 3 to be passed from the proximal to the distal end of the working element 11, within the bore of the inner sheath 1. The aperture 13 is offset, so that the telescope is located in the upper quadrant of the support guide 80 to make room for the electrode assembly 7.

The electrode assembly 7 can be inserted down the support guide 80 from the distal end thereof, and through a second aperture 15 in the sealing block 6. A plug portion 17 is provided at the proximal end of the electrode 7, and this plug portion is received within a socket located within the aperture 15, to be described below in further detail with reference to FIG. 4. A telescope clip 81, provided on the electrode 7, ensures radial alignment of the electrode with respect to the working element 11, and this may be enhanced with other features such as a beveled end face (not shown) which may be present on the plug portion 17. Other features, such as detents or other locking mechanisms (not shown) may be present between the plug portion 17 and the socket located within the aperture 15, in order to ensure longitudinal alignment and complete engagement therebetween.

As can be seen in FIG. 4, the plug portion 17 consists of a single pin 19 including a plurality of contacts, as follows. An active electrode contact 21 is provided at the end of the pin 19, and a return electrode contact 23 is provided at the opposite end of the pin 19. Between the contacts 21 and 23 is an identification contact section 25 including identification contacts 27, 28, 29, 30 and 31. Each of the contacts are separated one from another by bands of insulating material 32.

The plug 17 is received within a generally cylindrical socket 33, including contacts 34 to 40 adapted to make electrical contact with the contacts on the pin, when the pin 19 is received within the socket 33. Active electrode contact 21 on the pin is adapted to mate with contact 34 of the socket, while return electrode contact 23 is adapted to mate with contact 35 of the socket. Similarly identification contacts 27, 28, 29, 30 and 31 on the pin are adapted to mate respectively with contacts 36, 37, 38, 39 and 40 of the socket 33. Contacts 34 and 35 of the socket are connected respectively by leads 41 and 42 to the active and return outputs of the electrosurgical generator 10. Contacts 36 to 40 of the socket are connected by means of leads 43 to 47 to the identification input of the generator 10, each one of leads 43 to 47 also including a capacitor 48 to 52, the capacitors 48 to 52 each being of a different non-zero value. The pin 19 has a first radius along most of its length, but a reduced radius in the region of contacts 27 & 29.

The operation of the identification system is as follows. When the electrode 7 is inserted into the resectoscope 11, the pin 19 is received within the socket 33. This means that the contacts 21 and 23 make electrical contact with contacts 34 and 35 to provide the electrical pathway for the active and return electrodes of the bipolar electrode assembly 7. In addition, contacts 27, 28, 29, 30 & 31 in the identification section 25 of the pin 19 try to mate with the corresponding contacts 36 to 40 of the socket. However, depending on the type of electrode assembly 7 being used, the reduced radius in the region of contacts 27 & 29 means that pin contacts 27 & 29 do not mate with their respective socket contacts 36 & 38. This means that, for this particular example, identification contacts 28, 30 and 31 will make electrical contact with socket contacts 37, 39 & 40, but identification contacts 27 & 29 will not make electrical contact with socket contacts 36 & 38. This means that when the generator sends a signal to initiate the identification of the electrode assembly 7, capacitors 49, 51 & 52 will be sensed as being present, while capacitors 48 & 50 will not. The total value of capacitors 49, 51 & 52 is determined by the generator 10, and this serves to identify the electrode assembly 7 as being of a particular type (“Type A”).

An electrode assembly of a different type to that or electrode assembly 7 (“Type B”) will have a different contour such that different regions, say for example in the region of contacts 28 & 30, will have the reduced radius. In this case the generator 10 will sense capacitors 48, 50 & 52, but not capacitors 49 & 51. The total of capacitors 48, 50 & 52 will give a different value to that of capacitors 49, 51 & 52, thereby identifying the electrode assembly as being a Type B assembly as opposed to a Type A assembly. By providing the reduced radius in the region of different combinations of the identification contacts, many different types of electrode assembly 7 can be identified by the generator 10, and distinguished one from another so that the appropriate settings can be employed for each appropriate electrode type, and inappropriate electrode types can be rejected.

By making the active electrode contact 21 at one end of the pin 19, and the return electrode contact 23 at the opposite end of the pin, this ensures that power is only supplied to the electrode assembly 7 when the pin 19 has been fully and correctly inserted within the socket 33. The generator 10 can check that both the active contact 21 has made electrical contact with its associated contact 34, and that the return contact 23 has made electrical contact with its associated contact 35, before the identification process is carried out. This ensures that no false identifications are made by the pin 19 being only partially inserted within the socket 33. It is only when the pin has been determined as being fully inserted, and the identification process has been successfully completed that power is supplied to the electrode assembly 7. The identification process can be frequently repeated by the generator 10, to ensure that the status has not changed during use.

In FIG. 4, the socket contacts are shown as being provided along only one side of the socket. However, it is equally feasible to provide socket contacts along both sides of the socket, in order to save space with the socket design. Either the socket contacts on one side are staggered with respect to the contacts on the other side, or the socket contacts are located opposite one another. For each pair of oppositely located socket contacts, the pin contacts can be designed to contact either the contact on one side or the other, to contact both simultaneously, or to contact neither depending on the shape or layout of the pin contacts. In this way a large number of different combinations can be provided within a very small space.

FIG. 5 shows an alternative design of plug and socket, in which a plug with a plurality of pins is used. Plug 17 is provided with 5 pins, 60 to 64, which pins are received in channels 65 to 69 provided in socket 33. Each channel has contacts 70 to 74, the contacts being located only at the far end of each channel. As can be seen in FIG. 5, most of the pins are sufficiently long so as to make contact with the contacts 70 to 74, pin 60 and contact 70 being the connection to the active electrode of the bipolar electrode assembly 7, and pin 64 and contact 74 being the connection to the return electrode of the bipolar electrode assembly 7. Pins 61, 62 & 63 are identification pins, and one or more of these pins (in this case pin 62) is of a shorter length so as to deliberately fall short of making contact with its respective channel contact (in this case contact 72). As before, the contacts are connected to the generator 10 by way of leads with capacitors attached thereto (omitted in FIG. 5 to promote clarity of other features). In this way, the generator 10 can determine which identification pins are in contact with their respective channel contacts, and hence identify the type of electrode assembly attached to the resectoscope.

If more pins are provided, an even larger number of different combinations can be identified, allowing an even larger number of different components to be distinguished. As before, as an alternative to providing one or more pins shorter than the others, the pins can all be of the same length (sufficient to reach the contacts 70 to 74), but the ends of one or more pins can be masked with an electrically insulating covering so as to prevent electrical connection between that particular pin and its associated socket contact.

In the embodiment of FIG. 5, pin 62 is effectively redundant. If it is preferred that each of the pins still performs some function (such as connecting at least one of the electrodes of the electrode assembly to the electrosurgical generator), each pin can be provided with at least two conductive sections, say a first proximal section and a second distal section. These two sections are separated by an insulating middle section, and each section is provided with its own lead. Correspondingly, each socket channel is provided with two sets of contacts, a proximal set of contacts (for connecting with the proximal section of each pin) and a distal set of contacts (for connecting with the distal section of each pin). In this way, for example, the distal contacts can be used for identifying the instrument, and the proximal contacts can be used for supplying electrosurgical voltages or data between the electrode assembly and the generator. In this way, each of the pins is still functional, even if it only makes contact with one of the two sets of contacts in each channel. Alternatively, a system can be envisaged consisting of a combination of FIGS. 4 & 5, in which multiple pins are provided, each with multiple contacts as described. This provides a large number of available contacts, both for identification purposes and also for power supply to the electrode assembly.

Further different arrangements will be apparent to those skilled in the art, without departing from the scope of the present invention. For example, as an alternative to the capacitors described above, other components such as resistors or even digital memory devices can be used to identify the particular combination of the plug when it is engaged with the socket. The identification components, whether they are resistors, capacitors or other components, can be provided associated with the electrode assembly, or alternatively present in a cable or other connector. Although the system described above is employing an electrode assembly being connected to a resectoscope, the same arrangement can be employed to connect other electrosurgical instruments to an electrosurgical generator. For example, the plug and socket arrangement described above can be used to connect an electrosurgical instrument to an associated cable, the cable into a generator, or one part of a two-part instrument to the other (for example, where an instrument employs a single-use component with a re-usable handpiece).

1. An electrosurgical system including a first component and a second component detachably connected one to the other, the first component being provided with a socket including a plurality of contacts, and the second component being provided with a plug including one or more pins, the arrangement being such that when the plug is received within the socket the one or more pins make electrical contact with a particular combination of the plurality of contacts, the electrosurgical system being provided with means for identifying the second component by determining the combination of contacts in which the one or more pins are in electrical contact, wherein the plug includes at least one pin provided with different longitudinal portions, wherein each longitudinal portion along the pin is either provided with a first condition such that it makes electrical contact with its respective contact within the socket, or a second condition such that it does not make electrical contact with its respective contact within the socket, and wherein the first and second conditions are constituted by a different dimension for the longitudinal portions.

2. An electrosurgical system according to claim 1, wherein the first and second conditions are constituted by a radius for the longitudinal portions.

3. An electrosurgical system according to claim 1, wherein the first and second conditions are constituted by the presence or absence of a cut-out provided on the pin.

4. An electrosurgical system according to claim 1, wherein some or all of the plurality of contacts within the socket are connected to an identification component.

5. An electrosurgical system according to claim 4, wherein the identification component is a circuit adapted to generate an identifiable characteristic signal.

6. An electrosurgical system according to claim 5, wherein the identification component is a memory device.

7. An electrosurgical system according to claim 6, wherein the memory device is a digital memory device.

8. An electrosurgical system according to claim 4, wherein the identification component is a passive electrical identification component having a parameter of a finite non-zero value.

9. An electrosurgical system according to claim 8, wherein the identifying means identifies the second component by determining the total value of the passive electrical identification components for those contacts within the socket to which the one or more pins are in electrical contact.

10. An electrosurgical system according to claim 8, wherein the passive electrical identification component is a resistor and the parameter is resistance.

11. An electrosurgical system according to claim 8, wherein the passive electrical identification component is a capacitor and the parameter is capacitance.

12. An electrosurgical system according to claim 1, wherein the first component is selected from the group comprising electrosurgical generator, cable, or electrosurgical handpiece.

13. An electrosurgical system according to claim 1, wherein the second component is selected from the group comprising cable, electrosurgical handpiece, or electrode assembly.

14. An electrosurgical system including a first component and a plurality of second components each capable of being individually connected to the first component, the first component being provided with a socket including a plurality of contacts, and the second component being provided with a plug including one or more pins, the arrangement being such that when the plug is received within the socket the one or more pins make electrical contact with a particular combination of the plurality of contacts, the electrosurgical system being provided with means for identifying the second component by determining the combination of contacts in which the one or more pins are in electrical contact, wherein the plug includes at least one pin provided with different longitudinal portions, wherein each longitudinal portion along the pin is either provided with a first condition such that it makes electrical contact with its respective contact within the socket, or a second condition such that it does not make electrical contact with its respective contact within the socket, and wherein the first and second conditions are constituted by a different dimension for the longitudinal portions.

15. An electrosurgical system including a first component and a plurality of second components each capable of being individually connected to the first component, the second components being provided in a plurality of different types of second component, the first component being provided with a socket including a plurality of contacts, and the second component being provided with a plug including one or more pins, the arrangement being such that when the plug is received within the socket the one or more pins make electrical contact with a particular combination of the plurality of contacts, the electrosurgical system being provided with means for identifying the type of the second component by determining the combination of contacts in which the one or more pins are in electrical contact, wherein the plug includes at least one pin provided with different longitudinal portions, wherein each longitudinal portion along the pin is either provided with a first condition such that it makes electrical contact with its respective contact within the socket, or a second condition such that it does not make electrical contact with its respective contact within the socket, and wherein the first and second conditions are constituted by a different dimension for the longitudinal portions.

16. An electrosurgical system including a first component and a second component detachably connected one to the other, the first component being provided with a socket including a plurality of contacts, and the second component being provided with a plug including a plurality of pins, the arrangement being such that when the plug is received within the socket the plurality of pins make electrical contact with a particular combination of the plurality of contacts, the electrosurgical system being provided with means for identifying the second component by determining the combination of contacts in which the plurality of pins are in electrical contact.

17. An electrosurgical system according to claim 16, wherein each pin within the plug is either provided with a first condition such that it makes electrical contact with its respective contact within the socket, or a second condition such that it does not make electrical contact with its respective contact within the socket.

18. An electrosurgical system according to claim 17, wherein the first and second conditions are constituted by a different length for the pins.

19. An electrosurgical system according to claim 17, wherein the first and second conditions are constituted by providing the pin with either an electrically conductive or electrically non-conductive contact portion.
